(19) United States
(12) Patent Application Publication
(10) Pub. No.: US 2004/0034284 A1
(43) Pub. Date: Feb. 19, 2004

(54) Patient initiated emergency response system

(76) Inventors: Thomas R. Aversano, Baltimore, MD (US); Dale R. Elkiss, Hanover, PA (US); James G. Palmer, Ellicott City, MD (US); Francis B. Weiskopf, Catonsville, MD (US); William L. Maughan, Pasadena, MD (US)

Correspondence Address: THE JOHNS HOPKINS UNIVERSITY, Applied Physics Laboratory, 11100 Johns Hopkins Road, Laurel, MD, 20723-6099, US

(21) Appl. No.: 10/410,682

(22) Filed: Apr. 10, 2003

Related U.S. Application Data
(60) Provisional application No. 60/371,327, filed on Apr. 10, 2002.

(51) Int. Cl. A61B 5/00

(52) U.S. Cl. 600/300

(57) ABSTRACT

A method and system for encouraging cardiac patients to seek prompt medical attention upon the onset of symptoms of an AMI or other serious heart problems that can be identified by ECG's or other similar measurements is disclosed. A patient is prescribed a personal monitoring module that includes the necessary equipment to monitor one or more parameters (e.g., heart rate, ECG, etc), and which has the ability to automatically transmit or “push” the data relating to these parameters to a central server. The personal monitoring module utilizes a novel harness to automatically situate the ECG leads in proper positions, and can be operated in different modes so that it can be used for training purposes and for routine data gathering, as well as be used in emergent situations to, when appropriate, automatically alert appropriate medical personnel and initiate the process of obtaining medical assistance for the patient. Healthcare professionals can access the central server to view the data and telecommunications links can be established so that the entire emergency team can be apprised of the information pertaining to the patient almost immediately, where appropriate.

### CROSS-REFERENCE TO RELATED APPLICATIONS

This application claims the benefit of prior filed U.S. provisional Application No. 60/371,327, filed on Apr. 10, 2002, and incorporated fully herein by reference.

### STATEMENT OF GOVERNMENTAL INTEREST

This invention was made with Government support under Grant No. LM13542 awarded by the National Library of Medicine. The Government has certain rights in the invention.

### BACKGROUND OF THE INVENTION

1. Field of the Invention

This invention relates to the field of patient monitoring and, more particularly, to the field of remote monitoring of cardiac patients.

2. Description of the Related Art

Death and disability from heart attacks or acute myocardial infarction (AMI) is an extraordinarily large public health problem. The problem is so significant that in 1991, the NIH launched the National Heart Attack Alert Program (NHAAP) as a means of educating the public about heart attacks and their prevention.

According to the program description of the NHAAP, it is estimated that there are 1.1 million heart attacks each year. In 1996, 476,000 people died from AMI, approximately 51 percent being male and 49 percent being female. More than one-half of these deaths occurred suddenly, within one hour of symptom onset, outside of the hospital setting.

Studies show that the greatest reduction in mortality from heart attacks occurs when patients are treated early, e.g., within one hour of the onset of symptoms. Life-saving techniques such as pharmacological (e.g., thrombolytic or clot-dissolving therapy) or mechanical (e.g., percutaneous transluminal coronary angioplasty or PTCA) intervention are very successful in reducing AMI mortality, particularly when administered within the first hour.

Acute coronary syndrome (ACS) is a term used to describe a patient who has chest pains or other symptoms which could indicate either unstable angina or AMI. ACS presents a problem for both patients and health care personnel, since the treatment of unstable angina does not necessarily require the extremely rapid response required for treatment of AMI. Thus, for example, a patient may be unwilling or not appreciate the need to immediately contact healthcare providers upon onset of chest pains, believing the condition to be unstable angina rather than AMI. A patient having an AMI may waste precious minutes while taking medication to control what they perceive to be angina.

Since quick emergency treatment results in reduced morbidity, one of the primary goals of the NHAAP is to reduce morbidity and mortality from AMI through rapid identification of AMI symptoms so that prompt treatment can be obtained. Despite the success of efforts to educate the public regarding the need for early heart attack care, a barrier remains between patients (particularly acute coronary syndrome patients) and entry into the emergency medical system. Embarrassment (concern that chest pains may only be a “false alarm” due to indigestion or other causes), fear (not wanting to face the reality of heart problems), seeking advice from non-emergency sources (a general physician, family members, etc.), a history of angina or diabetes (other causes of symptoms similar to AMI but which do not require such emergent treatment), and the age and sex of the person experiencing the symptoms (the elderly and females tend to avoid seeking emergency treatment) are all factors that can play into the delay of seeking emergency medical treatment upon the onset of symptoms such as chest pains.

The NHAAP has identified three phases where delay can occur in the identification and treatment of individuals with a potential heart attack, as follows:

Phase 1: Patient and bystander recognition of the symptoms and signs of AMI and their actions in response to these symptoms.

Phase 2: Pre-hospital action by emergency medical services providers, that is, in response to patients prior to their arrival at the hospital.

Phase 3: Hospital action by health-care providers at the hospital to identify and treat patients with the symptoms and signs of AMI.

The pervasiveness of relatively low cost, high powered computer systems and advances in telecommunications capability has led to several attempts to provide patients and healthcare providers with means to quickly transmit critical medical data, such as electrocardiogram (ECG) readings, from the patient to the healthcare provider as soon after symptoms occur as is possible. For example, SHL TeleMedicine Ltd. of Tel Aviv, Israel (www.shahal.co.il) and Aerotel Medical Systems Ltd. of Holon, Israel (www.aerotel.com) each provide products and services that enable an individual to transmit ECG and other medical data via the telephone to a monitoring center, where the transmitted data is analyzed and diagnoses can be made remotely from the patient.

While the above-described systems function adequately, significant user interaction is required. For example, using either system, the patient, upon experiencing symptoms, attaches sensors (ECG electrodes) to appropriate locations on his or her body and holds a recording/transmitting unit (containing additional sensors) to their chest, records the ECG readings, initiates a telephone connection between the patient location and the central monitoring location, and then physically holds the telephone device (land line receiver or cellular telephone) up to the monitoring equipment, and the sounds transmitted by the monitoring equipment (representing an analog version of the ECG waveform) are output via a speaker and are transmitted over the voice line, where they are received at the monitoring station and converted to digital format and then read by the monitoring center operator. Thus, manual operations are required by the user, and ambient noise in the environment of the user can be transmitted over the voice line, thereby increasing the potential for the transmission of inaccurate or faulty data.

In addition, these devices operate in a single mode that results in a nurse and/or a physician always responding to the patient regardless of the situation occurring that initiated the transmission of the data. Further, a patient using these systems has no way to check the data obtained by the ECG electrodes. Thus, to practice using the device, and when the patient is in the process of positioning and repositioning the electrodes to obtain the correct positioning, the data is always transmitted to the monitoring station, where the monitoring personnel must advise the patient as to whether or not the electrode positions are correct and are thus transmitting valid data. In addition, once data has been obtained using these systems, it is not retained in the patient equipment, but is instead fully transmitted and stored on the healthcare side, potentially leaving first responders, e.g., emergency services personnel (as well as the patient) without access to the data and an inability to transmit the data to anyone else.

Accordingly, it would be desirable to have an ECG system which minimizes user interaction, which enables practice use without involving healthcare professionals unless needed, which enables local storage of data even after transmission of the data to another location, and which minimizes the likelihood of the insertion of faulty data signals during transmission of the data to a central unit.

### SUMMARY OF THE INVENTION

The present invention is a method and system for encouraging cardiac patients to seek prompt medical attention upon the onset of symptoms of an AMI or other serious heart problems that can be identified by ECG's or other similar measurements. In accordance with the present invention, a patient is prescribed a personal monitoring module that includes the necessary equipment to monitor one or more parameters (e.g., heart rate, ECG, etc), and which has the ability to automatically transmit or “push” the data relating to these parameters to a central server. The personal monitoring module utilizes a novel harness to automatically situate the ECG electrodes in proper positions, and can be operated in different modes so that it can be used for training purposes and for routine data gathering, as well as be used in emergent situations to, when appropriate, automatically alert appropriate medical personnel and initiate the process of obtaining medical assistance for the patient. Healthcare professionals can access the central server to view the data and telecommunications links can be established so that the entire emergency team can be apprised of the information pertaining to the patient almost immediately, where appropriate.

### BRIEF DESCRIPTION OF THE DRAWINGS

FIG. 1 illustrates the overall system environment of the present invention;

FIG. 2 illustrates, conceptually, the patient equipment used by a patient of the inventive system from the patient location;

FIG. 3 illustrates the details of the server;

FIG. 4 is a flowchart illustrating an example of steps performed in a typical use of the present invention; and

FIG. 5 illustrates, conceptually, the novel electrode harness of the present invention.

### DESCRIPTION OF THE PREFERRED EMBODIMENT(S)

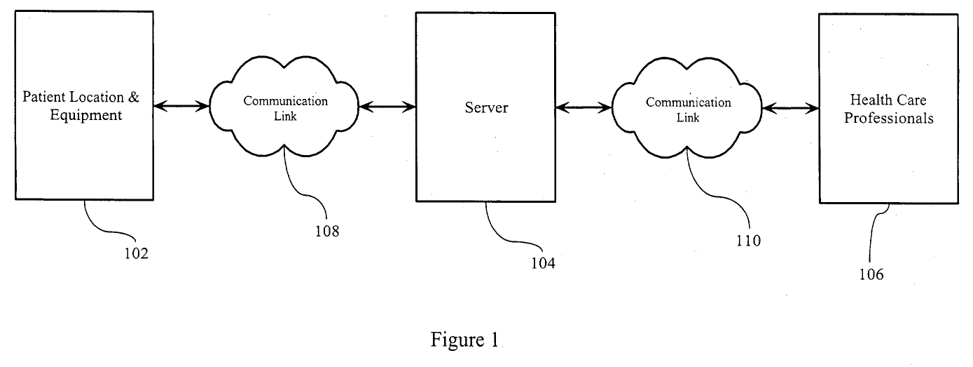

FIG. 1 illustrates the overall system environment of the present invention. Referring to FIG. 1, a patient location (e.g., a house) 102 has monitoring equipment available for use by the patient. A server 104 is connected to the patient location 102 via communication link 108. Further, healthcare professionals 106 are provided with a communication link with server 104 via communication link 110. This conceptual configuration allows for communication between the patient located at the patient location 102 and healthcare professionals 106, with, if desired, some or all of the communications being coordinated by the server 104 over the communication links 108 and 110.

Communication links 108 and 110 can comprise any means of data/voice communications. For example, communication links 108 and 110 can comprise landline telephone connections, wireless telephone connections, local area network (LAN) connections, connections via the Internet, and the like. For example, it is contemplated that certain health facilities may access the server via Internet connection while others may communicate via telephone lines and telephone data connections. Paging systems and wireless telephone and PDA devices are also considered to be potentially used with the system and able to communicate over the communication links 108 and 110.

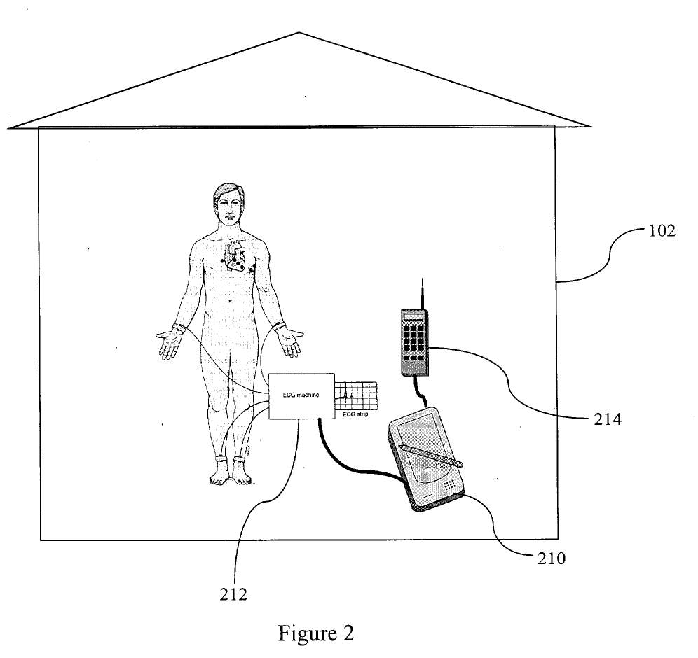

FIG. 2 illustrates, conceptually, the patient equipment used by a patient of the inventive system from the patient location 102. The general elements of the patient equipment are a processing device capable of processing ECG data, means for collecting ECG data, means for storing the collected and processed ECG data, and means for connecting to a telephone system (wireless or land line) for transmission of the data to server 104. Referring to FIG. 2, a processing device 210 is coupled to an ECG machine 212 and a telephone 214. In FIG. 2, a hand-held PDA is illustrated for processing device 210; a stand-alone ECG machine generating an ECG strip is illustrated for ECG machine 212; and a wireless telephone is illustrated for telephone device 214. It is understood that this is illustrated as an example only and it is contemplated that the device used by the patient can comprise a single device incorporating all four elements (processing, ECG collection, storage, and telecommunications), or can be combined in two or more devices. For example, the patient equipment can comprise a PocketView ECG device by Micromedical; a Hewlett-Packard/Compaq PocketPC PDA w/dual sleeve expansion pack; a Merlin C201 CDMA2000 PC datacard; the Sprint CDMA 2000 Network, and a separate telephone for IVR/teleconsultant dialog. Alternatively, the patient equipment can comprise the Micromedical PocketView ECG; a Toshiba PocketPC PDA; a Growell Telecom CF2031 CDMA2000 CF datacard; and integrated IVR/teleconsultant dialog. An additional alternative can comprise a Micromedical PocketView ECG; a Seamans SX56 XDA Pocket PC phone coupled to the AT&T GSM network. By using an integrated cell phone PDA, such as the Seamans SX56 XDA PocketPC phone, the design is simplified, particularly in terms of power management. CDMA 2000 versions of this phone will be implemented in the future, and the present invention is contemplated as including the ability to operate on these and other such networks.

Further, it is contemplated that a customized device can be created which is capable of doing the ECG measurements, the processing required and also be wireless and have cell phone capability, with the ability to transmit data over a standard modem in the device, with an acoustic coupler back-up. Any device or devices capable of performing the processing, ECG collection, storage, and telecommunications functions described herein can be used. Further, while conventional ECG leads may be used, in a preferred embodiment, a novel electrode harness is used, details of which are described more fully below.

Functionally, the patient equipment must be able to collect and store multiple ECG readings (ten 30-second ECG readings, for example) and connect to a server via telecommunication link to “push” the stored collection data to the server when desired and/or automatically, e.g., via FTP. To simplify the use of the device, in the preferred embodiment, the processing element of the system is configured to take the ECG readings, store them, and forward them to the server automatically, essentially upon the push of a button (e.g., an icon on a PDA), after the user has placed the ECG electrodes on the body.

It is also contemplated that the processing device will be configured to operate in multiple modes. For example, in a first mode, the device can be used to take and if properly configured, view, the ECG measurements without sending them to the server, unless it is desired. Visual and/or audible indicators are included in a preferred embodiment to provide indications to the user regarding the correct or incorrect placement of electrodes. This gives the user the opportunity, for example, to test the placement of the electrodes and take measurements to determine the proper location. The user can see the results of the ECG collection and determine if the electrodes are placed properly, without needing to send the data to the server for analysis by a technician or automated analysis tool. In other words, the user has the ability to test the ECG electrode connection without bothering the central office or other monitoring location.

A second mode can be configured to automatically store the data on the processing device and, if desired, automatically push the data to the server. This encourages the user to take periodic readings when not experiencing chest pains or other symptoms, thereby storing on the server (and locally) baseline measurements which can later be compared, if necessary, with measurements taken during a cardiac event. Further, by storing the baseline measurements locally on the PDA or other processing device (instead of forwarding it without locally storing it as is done in the prior art), the patient has the ability to later send the data to other interested parties, for example, to EMTs, when needed, as well as to review the data himself or herself. If desired, the device can also be configured to remind the user to take measurements, for example, by sounding an alarm on the processing device. This prompts the user to take a regular reading (e.g., weekly, monthly, etc.).

The device can be configured for a third mode, which is utilized when the patient is experiencing chest pains or other symptoms. In this mode, the device takes the ECG readings, sends them immediately to the server, activates a paging system to alert a medical professional that data is incoming for immediate analysis and, if desired, can also contact EMTs or other medical professionals to dispatch an ambulance to the patient's location. If desired, GPS coordinates can be transmitted with the ambulance request so that it will not be necessary to give address information, i.e., the location of the patient will be automatically ascertainable by the ambulance crew. However, other techniques of ascertaining the patient location, including Caller ID and verbal interaction with the patient, can also be utilized.

The above modes are examples only and it is contemplated that other modes of operation can be utilized in connection with the present invention. For example, automatic screening of medical data by software can be utilized, so that alerts are issued only when certain thresholds are met.

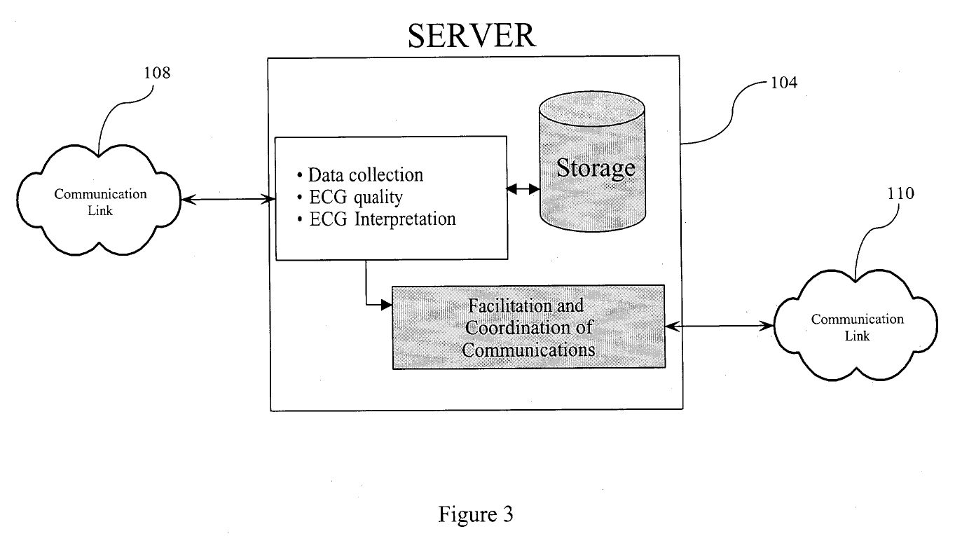

Details of server 104 are illustrated in FIG. 3. Server 104 serves important coordination and control functions of the present invention. Server 104 can comprise, for example, a Pentium III (or higher) workstation running Windows 2000 and capable of supporting Microsoft VB.NET Framework, and having a modem, network connection, and running an FTP server. Communication link 108 provides a path for data, voice, or any other communications required between the server and the patient location 102. Server 104 is configured to collect and store the data, automatically analyze the quality of the ECG data using existing ECG analysis algorithms such as the Marquette Analyzer. This enables interpretation of the ECG data (i.e., to perform a screening function so that “normal” ECG data is simply stored, while abnormal ECG data may trigger an alert). Further, server 104 must also be configured to facilitate and coordinate communications between the patient, the healthcare professionals, and the data needed by the healthcare professionals, which is stored on the server 104. Thus, communication link 110 is also connected to server 104 to provide a communication link (data, voice, etc.) between the server and the healthcare professionals 106 (see FIG. 1).

In a preferred embodiment, the server 104 is also IVR (interactive voice response) enabled so that both patients and healthcare professionals can communicate with the server in an automated manner over a standard telephone line or connection. This gives the patient the ability to input data to the server using touchtone or voice input so that data, in addition to the ECG data, can be gathered and stored by the server. Further, having IVR capability gives the healthcare professionals the ability to retrieve data and redirect data to other healthcare professionals as needed.

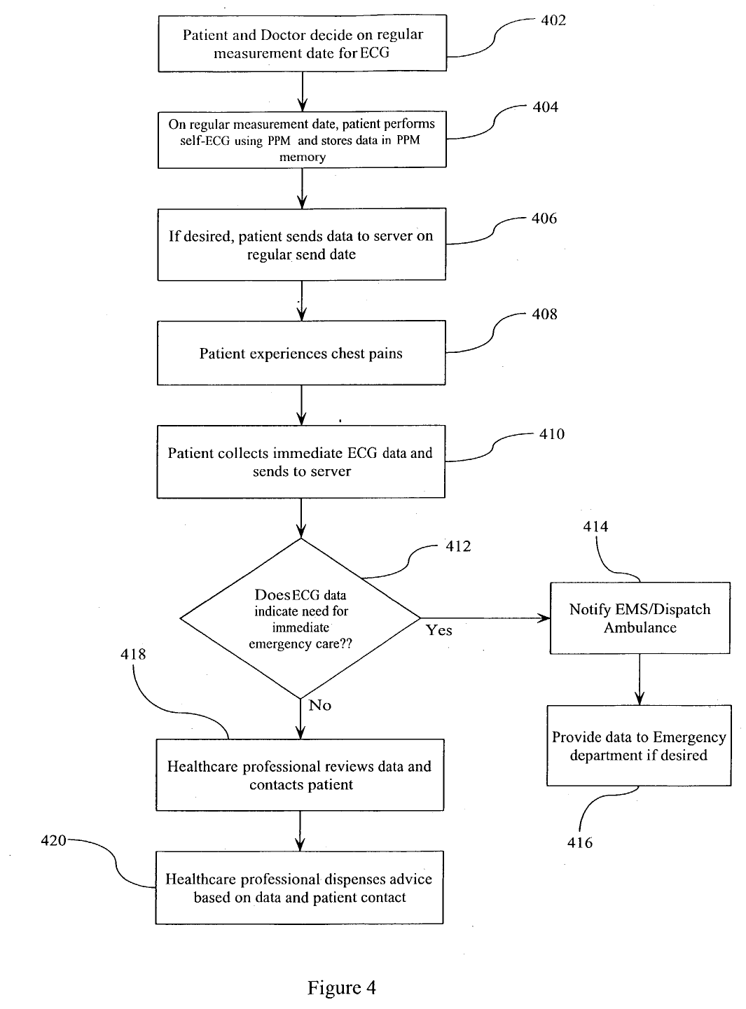

FIG. 4 is a flowchart illustrating an example of steps performed in a typical use of the present invention. It is understood that this flowchart is for example only and that numerous other operational modes are covered by the present invention.

Referring to FIG. 4, at step 402, a determination is made by the patient and the doctor that a regular measurement date for ECGs will be established, and the date (e.g., every Monday, once monthly, etc.) is decided upon. The interval/regular measurement date for ECGs is input to the patient equipment so that appropriate alarms or other indicators can be set, if the device is configured for this feature.

At step 404, on the regular measurement date, the patient performs a self-ECG using the personal patient monitor and stores the data in the personal patient monitor memory. The first measurement can be made by a physician to establish a baseline measurement, either with the patient equipment or with separate office-based equipment. Alternatively, the patient takes measurements using the personal patient monitor and establishes the baseline data set in this manner, which can then be stored locally on the personal patient monitor and saved there indefinitely, if desired.

At step 406, if desired or requested by the doctor, the patient sends the data to the server on a regular send date. This can be simultaneous with the regular measurement date, or several ECG readings can be stored and transmitted on a subsequent regular send date.

At step 408, the patient experiences chest pains, and at step 410, the patient immediately collects ECG data and sends it to the server. As noted above, this can be automatically instituted by, for example, the user selecting an emergency mode (e.g., mode 3 described above), which will automatically, once the electrodes have been put in place and the system activated, take the measurements and forward them to the server, while simultaneously sending notice to a healthcare provided to look for the incoming data. This notice can be performed using paging devices, telephones, or any other means of communicating with the healthcare provider.

At step 412, a determination is made as to whether or not the ECG data transmitted by the patient indicates a need for immediate emergency care. This would be appropriate in situations where the ECG data indicates or suggests the possibility that an AMI is occurring. If, at step 412, it is determined that emergency care is necessary, the process proceeds to step 414, where emergency medical services (EMS) are contacted and, if needed, an ambulance is dispatched to the patient location. If desired, GPS data can be transmitted with the ECG indicating the location of the patient; alternatively, address information can be gleaned by other means, such as, for example, patient data. Further, at step 416, the ECG data can also be provided to (or made available to) an emergency department which will be receiving the patient via ambulance and/or who will be otherwise giving medical attention to the patient.

If, at step 412, it is determined that there is no indication that immediate emergency care is necessary, the process proceeds to step 418, where a healthcare professional reviews the data and contacts the patient. Typically, this contact will be by telephone so that the patient receives immediate attention by the healthcare provider, although it is contemplated that other non-emergency means can be used, for example, email. At step 420, based upon the review of the data and discussions with the patient, the healthcare professional dispenses advice, for example, directing the patient to seek medical attention during normal business hours and/or to begin a predetermined medical regimen (e.g., to take medication that has been previously provided to the patient for situations indicated by the ECG data).

Using the above system, the patient learns to use the equipment proficiently, since the patient is being monitored, and thus using the equipment, on a regular (weekly, monthly, daily, etc.) basis while at the same time establishing baseline data that can be compared with data being taken during a cardiac event. This information can be extremely beneficial to the healthcare provider and the fact that it is “near real time” can reduce the amount of time taken by the patient in getting medical attention. Further, by using the device on a regular basis, the patient may be less inhibited about sending the data when it is needed.

Although not described in the flowchart of FIG. 4, the patient may also on a regular basis take “practice readings” which are displayed locally for the user on a regular basis but which are not transmitted to the healthcare provider/server. This further assists the user in becoming comfortable with the equipment and its use and increases the likelihood that the user will send meaningful data to the healthcare provider at the appropriate times.

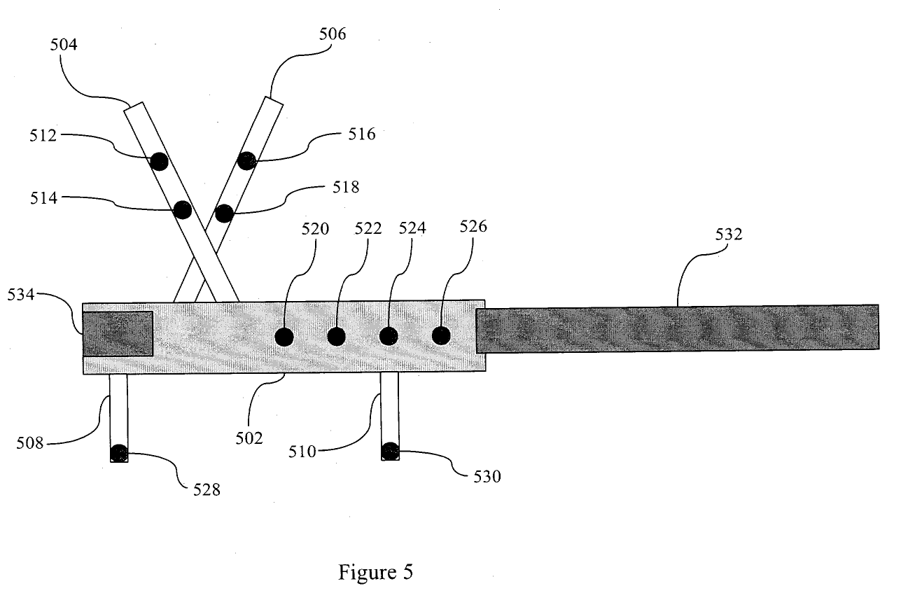

As noted above, in a preferred embodiment, a novel electrode harness is utilized to properly diagnose myocardial infarction or acute coronary syndrome; to simplify the use of the ECG device for the patient, and maximize the likelihood that proper readings will be obtained; to reduce noise induced into the system; and to provide reusability to thereby increase the cost-efficiency of the system. FIG. 5 illustrates, conceptually, the novel electrode harness of the present invention. Referring to FIG. 5, the basic elements of the electrode harness of the present invention comprise an adjustable elastic chest belt 502, adjustable elastic shoulder straps 504 and 506, and hanging hip electrode straps 508 and 510. The harness is worn essentially similar to a brassiere, with the chest belt 502 being positioned just under the nipples for males and just under the breasts for females.

The adjustable elastic chest belt 502 can include a pouch for holding wiring harnesses, connectors, and other hardware, and it also provides a conduit through which electrode wires can be routed. In addition, the V3 through V6 electrodes 520, 522, 524, and 526, respectively, are attached to adjustable elastic chest belt 502 such that, when the device is being worn, they are in contact with the patient's body in such a way that the ECG readings can be obtained. The Velcro strap 532 and Velcro mating piece 534 allow the chest belt 502 to be adjusted around the user and hold the electrodes in place in such a manner that they can be used to take the ECG readings.

The adjustable elastic shoulder straps 504 and 506 each hold and provide contact pressure for V1 and V2 electrodes 514 and 518 as well as two shoulder electrodes 512 and 516.

In a preferred embodiment, the electrodes are nickel-plated brass for increased conductivity and because of its anti-corrosion properties. Any electrodes can be used; however, the use of nickel-plated brass increases conductivity and reduces corrosion of the electrodes, a significant issue in that the leads are intended to be reusable. Applicant is unaware of any existing nickel-plated brass electrodes but has determined that such electrodes can be fabricated using nickel-plated brass cover buttons available from Prym-Dritz Corp. with a 4-40 machine screw connected to it. A foam spacer can be placed over each machine screw (between the button and the fabric harness) to provide increased contact pressure. The machine screw is punched through the fabric harness (the location can, if desired, be customized to fit the needs of a particular wearer), and an ECG wire is attached to the machine screw inside the harness. The material used can be, for example, nylon pack cloth available from Top Value Fabrics, Inc. and standard Velcro and zippers can be used for fasteners. Obviously this is just an example, and any materials can be used which allow the harness to be simply and easily and adjustably placed on the body of the user and which will situate the electrodes in the appropriate locations as shown in FIG. 5.

Although not limited to these elements, the electronics pod and wiring can comprise the PocketView electronics pod by Micro Medical.

The harness is worn in such a manner that the V3 through V6 electrodes that are situated in the adjustable elastic chest belt 502 are located in the proper orientation for ECG data gathering when the arms are through the shoulder straps such that the shoulder straps 504 and 506 criss-cross the chest. The user adjusts the Velcro so that the chest belt is snugly attached to the user's body in the position described above. The orientation of the V1, V2, and two shoulder electrodes in the shoulder straps 504 and 506 places them in the appropriate location for these leads.

Hanging hip electrode straps 508 and 510 dangle off of the chest belt 502 and have attached thereto electrodes 528 and 530. To maintain these electrodes against the user's body, the user is instructed to tuck the electrodes into the waistband of pants being worn by the user, thereby placing the electrodes against the hip. The electronics pod (not shown) includes a coupling wire that can be interfaced with the computer and/or PDA using an RS-232 or other suitable interface. Specifically, the coupling wire uses voltage levels and control signals that conform to the EIA (Electronic Industrial Association) RS-232C standard. In one embodiment, a DB-9 connector is used, in alternative embodiments, direct wiring to a dedicated device with similar functionality as a PDA can be used; alternatively, a non-standard connector with a PDA or PDA/cellular telephone convergence device can be used. Further, it is understood that Blue Tooth wireless technology can be utilized instead of a coupling wire.

Using the harness of the present invention, a non-medically trained patient can easily place the electrodes in the proper location simply by putting on the harness in a manner similar to putting on an article of clothing. No gels are required; the user simply puts on the device and takes the readings.

### EXAMPLE

The following describes an example of a system constructed and operating in accordance with the present invention. The example system is referred to herein as the “Patient-Initiated Emergency Response System” or “PIERS”. The functionality built in PIERS reduces barriers to high-risk patient involvement with emergency medical services by a behavior program that actually involves the patient in the system. A first operating mode of the PIERS provides for periodic system operability checks and practice in system operation for the patient. The results of the operability check are provided to the patient in a formal manner that allows the patient to assess the operability of the equipment as well as how well the patient can use the equipment. This mode directly addresses elements concerning relapse prevention, feedback and self-monitoring behavior. A second operating mode of PIERS provides for periodic collection of medical data as well as an interim evaluation of ECGs. The medical data is stored to provide a baseline for the patient in a future medical situation and the ECG data supports long-term monitoring of the patient's cardiac health. This mode directly addresses feedback about progress, modeling a desired behavior, and self-monitoring behavior. A third operating mode provides for easy and direct access to medical expertise and services at the onset of chest discomfort. This mode directly addresses feedback about progress and self-monitoring behavior.

One of the primary goals of PIERS is, in effect, to bring critical elements of the emergency department to the patient. This is done in three ways: at-home (or wherever the patient may be) 12-lead ECG measurements at onset of chest discomfort; cardiologist interpretation of ECG reading and patient history; and EMS services alerted to the patient's condition, medical history and location.

The PIERS addresses EMS service factors in three ways: it lowers barriers to engaging EMS services because of multi-mode operation; it provides patient medical history information, including current medications, and comparative ECGs to the EMS providers to facilitate an EMS service call; and it directly links cardiac medical expertise with patient and EMS providers.

The Emergency Medical Services (EMS) in the United States are organized by states or local jurisdictions (e.g. county or municipality), and consequently, are subject to a wide range of regulations and controls. The net effect is that the protocol used by EMS providers can vary dramatically from location to location. Thus any system designed to bring the Emergency Department (ED) to the patient and decrease the barriers to using EMS in the case of an acute coronary syndrome (ACS) must be adaptable to accommodate local EMS protocols. The PIERS satisfies this requirement since the hardware/software installation can be configured for many arrangements or operating protocols and many arrangements of participants.

The basic concept of the system is to bring critical parts of the Emergency Department to the patient in a simple, rapid, reliable, inexpensive, non-threatening way that would lower the barrier to entry into the medical treatment system. By minimizing embarrassment and fear, by not requiring transportation to a specialized facility at which time and “face” may be lost, by serving, in effect, as an objective reviewer, this system reduces the time between symptom onset and treatment application for patients with ACS. Such a system leads to fewer false-positive trips to the emergency department, as well. PIERS is integrated seamlessly with the current EMS and 911 system and does not become an additional cause of delay between symptom onset and treatment.

In the absence of on-going acute ischemia or available provocative tests, coronary artery disease is detectable only by history. Both the past medical history, particularly cardiac risk factors, and the current history of a syndrome involving chest discomfort are key elements that provide clinical clues to an ACS. In the presence of on-going symptoms, the ECG is a simple, inexpensive, objective test that can often detect cardiac ischemia. The emergency department procedures for the initial evaluation of the ACS patient include past and current history, and a comparison of a past with a current ECG.

Two distinct, but related groups of patients at risk for developing ACS, including acute myocardial infarction, recognized are: 1) high risk patients—those with known coronary, peripheral or cerebrovascular atherosclerosis, and 2) the general population.

As an initial step, this system provides patients at high-risk for developing an ACS with the capability of transmitting current and past historical information, responses to questions that are indicators of ACS, baseline ECG, and current ECG from any telephone to a central facility which is available immediately at all times. This capability will be provided by a compact device the patient carries at all times. The device will be prescribed through a physician, who identifies the patient as being in the high-risk category. The information transmitted by the device is processed by a decision support module that will immediately activate EMS providers if an AMI is in process. All information is transmitted to a Cardiac Teleconsultant who interprets the ECG in combination with the medical history. After interpreting the information, the Cardiac Teleconsultant can direct the patient to report immediately to a Chest Pain Evaluation Center, to obtain an appointment with a physician as soon as possible or the EMS provider could be dispatched, if automatic dispatch has not already occurred.

For the general population, a Patient's Personal Module (PPM) will be made available at selected public places, such as pharmacies, sports arenas, etc., where, with the help of trained personnel, the ECG my be reported and evaluated by the PIERS Cardiac Teleconsultant. In addition, to serve those who cannot access the ECG module of the PIERS, data collected by the PIERS can be used to evolve a computerized, telephone-accessible decision support system to field calls. For example, the patient can have a touchtone telephone “dialog” with an automated interactive voice response system. This PIERS concept will be realized by the system with the following capabilities:

Highly portable, patient monitoring and data handling device for high-risk patients

device will store past medical history, current medications and baseline ECG

device will obtain and store current ECG

device will store ACS indicator questions and patient responses

device will be capable of transmitting stored information and current history to a central computer, physician and/or treatment facility via phone line

device will allow for patient interaction via the same phone line used to collect current history

Integration of monitoring, data handling and medical interpretation functions within a clinical pathway that provides continuity of patient care between emergency medical technicians and the hospital emergency department or Chest Pain Center

Ongoing collection of outcome data will be used to continuously evolve an improved automated decision support system. In essence, historical and ECG data will be used to risk stratify patients and define the best treatment pathway with increasing levels of automation, as the data permit and as the system evolves. This future decision support system will be based on history derived from patient dialog and will be adapted for both high-risk patients and for the general population.

The PIERS can be configured for different sets of medical providers. Possible participants include:

Patient

The typical patient is at high risk for ACS and has been provided with a PPM by his physician. The patient uses the device in accordance with prescription. The prescription will require the patient to use the PPM in periodic system checks (Mode 1), periodic data transmission (Mode 2), and immediate ECG reporting in the event the patient experiences chest discomfort, or other AMI symptoms (Mode 3). The patient and a person who might frequently be with the patient will receive training in PPM operation for all modes, and the circumstances and symptoms for which the patient should initiate Mode 3 operation. Other forms of training, including written materials, self-help materials, videos and internet-based training will supplement this initial training.

Personal Physician

A physician will likely prescribe the service for a patient and initialize the patient device. The personal physician will initialize the device with patient history and a baseline 12-lead ECG for the patient using the device. The training personnel at the personal physician's office will provide initial patient training in using the device and will be point-of-contact for patients who have questions and/or problems associated with device operation. The personal physician will receive all ECGs obtained during Mode 2 and 3 operation. The personal physician is responsible for managing the content of the data stored on the device and specifies how often the patient exercises Mode 2 operation.

Service Technician

The Service Technician is responsible for maintaining the system and assisting patients, when necessary, to assure trouble-free operation. During Modes 1 and 2 operation, the Service Technician responds to automatically or manually initiated alerts which indicate a system problem, verifies that the system provides necessary instructions to the patient (or personally instructs the patient, if necessary), checks the quality and timeliness of transmitted data, and verifies that the system forwards the transmitted data to the Personal Physician.

Cardiac Teleconsultant

The Cardiac Teleconsultant receives ECG data, ECG processing results, patient history and responses to automated questions from the system, interprets the data, speaks with the patient, and determines the next steps in the patient's care. The Cardiac Teleconsultant will alert EMS for necessary patient transport and immediate interventions (with the option of patient information forwarding to the EMS), as well as forward the patient data to the ED. A record including the patient name, date of call, relevant historical data (answered by touchtone) and diagnosis and disposition (entered by Cardiac Teleconsultant) will automatically be routed to the Personal Physician.

Emergency Medical Services

EMS receives a dispatch from either the Cardiac Teleconsultant or the patient (via 911). If an ECG has been obtained from the system, the Cardiac Teleconsultant will also advise EMS of diagnosis and recommended interventions during transport. EMS will alert the ED physician of an incoming patient.

Emergency Department Physician

The Emergency Department physician will be notified by EMS of a cardiac patient in transport. Any current history or ECG data obtained by the system will be available as well as a diagnostic summary from the Cardiac Teleconsultant.

As noted above, the preferred embodiment of the Patient Initiated Emergency Response System (PIERS) has at least three distinct modes of operation for the patient to interact with the system. The overall concept of operation for the PIERS for each of the operating modes is summarized below, as is the framework for the PIERS operation.

Event Sequence

Each use of the PIERS involves a sequence of events. Time is not a critical parameter for Modes 1 and 2 of the exemplary PIERS described herein, since these modes are not associated with the presence of chest discomfort. However, it is critical for Mode 3 operation. The general sequence of events in this example system, regardless of mode, can be broken into six steps:

The sequence is initiated when the patient is prompted to use the system (Modes 1 and 2) or the patient senses chest discomfort (Mode 3);

Patient puts on electrode patch and enables ECG sampling

Patient (or system, automatically) places telephone call to PIERS;

System processes patient data;

The system generates a response to a call:

1) Cardiac Teleconsultant reviews data and, if necessary, dispatch EMS personnel to patient (Mode 3); or,

2) System analyzes PPM operability and reports to patient and personal physician (Mode 1); or

3) System collects ECG and possibly other data for interpretation and storage on the patient's ECG device (Mode 2); and

Patient meets periodically with personal physician to maintain ECG unit information.

This general sequence of events is used as a reference when discussing the operating modes.

Summary of Operating Modes

What follows is a brief discussion of 3 possible operating modes in which the patient could use the system. These 3 modes are meant to provide an inclusive relationship between the high-risk patient and the medical services and to break down the barriers (cost, unfamiliarity with use, public attention, consumption of services, lost time) between the time a patient senses discomfort (Event 1), and the patient places a telephone call to PIERS (Event 2). To avoid replication, those steps common to all modes are described first.

Common to All Modes

When the patient chooses to operate the system, the operating mode on the PPM (default is Mode 3) is selected. For all modes, the patient attaches the ECG electrodes as previously instructed, activates data collection, and responds to questions stored in the PPM. The PPM will then retrieve patient history from memory (all modes) and initiate an ECG sampling and recording of 60 heartbeats. The patient initiates a phone connection to the System Server at a designated regional location. The history and recorded ECG data are transmitted with continued ECG sampling up to the number of desired beats. The System Server announces status at the end of the interaction.

If the ECG recording was not successfully received by the System Server, the patient will be prompted with questions and instructions to assist in using the ECG. If the ECG is still not successfully transmitted, additional (mode dependent) questions can be provided to obtain needed information and, if necessary, a maintenance action will be planned and, if necessary, EMS will be notified.

The PPM is typically connected via analog modem to an auto-answer modem bank supported by a rotary telephone switch at the System Server. The modems are capable of simultaneous voice and data transmission, so the patient can receive voice messages from the phone while the phone line is transferring digital information. Any method of connecting to the server for data/voice communication is contemplated by the present invention.

Mode 1: Operability Check and Practice Use

The patient is encouraged to practice using the system and to verify its operation. The process is totally anonymous and totally automated (other than patient actions); consequently, minimal consumption of resources (i.e., minimal cost) and minimum patient visibility or effort and consequently minimal barriers are incurred. The System Server verifies that parts of the ECG unit are operating and that the data can be successfully transmitted to the System Server. The patient is alerted to any performance issues or malfunctions via immediate feedback (display and/or recorded voice message, telephone call and/or mail). A scenario for Mode 1 operation is shown in Table 1. The two left-hand columns (“min” and “max”) estimate the minimum and maximum time between successive events in the scenario. Note that time is not critical in the Mode 1 scenario. The third column (“Event”) lists the events in the scenario. The last column (“Notes”) contains explanatory material where appropriate.

TABLE 1
Mode 1 Scenario
T + (HR:MIN)

| Min | Max | Event | NOTES |
|---|---|---|---|
| 00:00 | 00:00 | Calendar prompt to | Calendar maintained by ECG unit or |
| | | exercise ECG unit | server |
| | | | Exercise the maximum amount of |
| | | | circuitry possible |
| 00:05 | 48:05 | Activate unit with | |
| | | leads for patient | |
| | | training | |
| 00:32 | 48:15 | Electrodes attached | Electrode Reusable |
| | | by patient & ECG | First-level data validation by ECG |
| | | recorded | device |
| 00:31 | 48:30 | Patient responds to | Use buttons on PPM |
| | | automated questions | |
| 00:30 | 48:25 | Patient connects to | Direct connect to phone line with |
| | | communication | future wireless option |
| | | service | |
| 00:32 | 48:05 | Data Transfer to | ECG & history data, question |
| | | System Server | responses |
| 00:35 | 48:15 | Data validity checks | Kinds of checks include device mal- |
| | | | function, bad comms, battery low, |
| | | | data corrupted, other |
| | | | Service technician interrupts if mal- |
| | | | function indicated or follow-up call |
| | | | added to technician work list |
| 00:36 | 48:16 | Automated ECG | |
| | | interpretation | |
| 00:37 | 48:17 | Automated Re- | LCD message display and recorded |
| | | sponse to Patient | voice |
| | | | Contact Service Technician if unit |
| | | | needs repair |
| 12:00 | 96:00 | Report Results | Automated Service log maintained |
| | | | for Service Technician |

Mode 2: Periodic Health Status Check

For the general high-risk patient and for those with certain medical conditions, the patient is requested to periodically use the system to update the patient's ECG report and recent patient history. Examples of such historical features are chest pressure (character, frequency, level of exertion required to provoke, episodes per week, etc.), dyspnea, palpitations, syncope or near syncope, etc. Mode 2 operation is semi-automated; the system server receives patient history data and a current ECG from the ECG unit over the communication service. The System Server does data checking for validity. Any invalid indicators result in an alert to the Service Technician who will assist the patient as required (during normal working hours) or a follow-up call is added to the technician's worklist. Once accepted, the ECG waveform is interpreted by an algorithm such as the General Electric/Marquette algorithm. The assembled data is sent to the personal physician to interpret the information. When appropriate the patient visits the personal physician who has responsibility of updating patient's medical history and ECG data stored on the PPM during the office visit. The personal physician's office is also responsible for keeping medical records necessary for medical management, insurance and legal purposes. Table 2 provides a scenario for Mode 2; the column headings are identical to Table 1 and, as in Mode 1, time is not critical other than convenience to the patient.

TABLE 2
Mode 2 Scenario
T + (HR:MIN)

| Min | Max | Event | NOTES |
|---|---|---|---|
| 00:00 | 00:00 | Calendar patient | Calendar maintained by ECG Unit or |
| | | prompt | server |
| 00:30 | 48:00 | Activate unit with | |
| | | leads for medical | |
| | | purposes and patient | |
| | | training | |
| 00:32 | 48:15 | Electrodes attached | Electrode Reusable |
| | | by patient & ECG | First-level data validation by ECG |
| | | recorded | device |
| 00:34 | 48:45 | Patient connects to | Initially, direct connect to phone line |
| | | comms | Use of voice over IP (simultaneous |
| | | | data and voice) |
| | | | Patient interview via controlled voice |
| | | | recordings |
| 00:36 | 48:50 | Data transfer to | Data validity checks |
| | | system server | Data retransmissions, if needed |
| | | | Service technician interrupts if mal- |
| | | | function indicated or follow-up call |
| | | | added to technician work list |
| 00:36 | 48:16 | Automated ECG | |
| | | interpretation | |
| 00:37 | 49:00 | Response to Patient | LCD message and voice interaction |
| 12:00 | 96:00 | Report Results | Forward to personal physician |

Mode 3: Symptomatic

This mode provides significant benefit when the patient is experiencing chest discomfort or other symptoms of concern but is reluctant to call 911. The patient uses the PPM to collect an ECG and then uses the communication service to call PIERS which receives patient history data including responses to programmed medical questions and current ECG waveforms. The System Server does the same data validity checking as in Modes 1 and 2 and initiates a request for the patient to retransmit the data set if appropriate. As in Mode 2, an ECG interpretation algorithm performs an ECG assessment. If the historical and ECG data clearly indicate AMI, then EMS is immediately dispatched without any additional human intervention. The patient's data are then forwarded to a Cardiac Teleconsultant who is connected with the patient for voice interaction; the patient is informed EMS is on the way. In the event the historical and ECG data do not clearly indicate AMI, then the patient's data are forwarded to a Cardiac Teleconsultant for review and the patient is placed in direct voice contact with him. The Cardiac Teleconsultant then executes appropriate action. Possible interpretations include:

1. Priority Event—EMS will be immediately dispatched. The patient will be informed that the EMS is being sent and he may be advised to keep the system active for continued real-time monitoring. When EMS is dispatched, the patient data, such as demographics, history, medications, and baseline and current ECG can be transferred to the EMS service. Once a hospital ED is selected, the history data as well as the ECG data are made available to the designated ED.

2. Non-priority Event—The Cardiac Teleconsultant concludes that the ECG and historical data do NOT indicate that a life-threatening event is in progress. The patient is advised to call 911 if he believes that emergency treatment is needed. He is further advised to seek medical attention according to a number of scenarios detailed in the medical requirements document. There are a number of possible outcomes besides AMI: a) life-threatening but not AMI; b) unstable angina—needs EMS dispatch; c) crescendo angina—needs to see physician today or come to ED today (Cardiac Teleconsultant may need to make appointment on-line); d) change in angina but not particularly alarming (needs to see primary physician within 72 hours); non-cardiac and non-emergent (see physician and appointment can be made by patient).

Table 3 provides a scenario for Mode 3 operation.

TABLE 3
Mode 3 Scenario
T + (HR:MIN)

| Min | Max | Event | NOTES |
|---|---|---|---|
| 00:00 | 00:00 | Chest discomfort | |
| | | event | |
| 00:01 | 00:15 | Patient acknow- | Depends on severity of pain & patient |
| | | ledges symptoms | Awareness |
| 00:02 | 00:30 | Patient activates de- | Use of voice over IP (simultaneous |
| | | vice | data And voice) |
| | | | Programmable, tailored current his- |
| | | | tory questions and patient response |
| | | | Caller ID immediately available to |
| | | | client Workstations |
| 00:05 | 00:45 | Electrodes attached | Reusability |
| | | by patient and ECG | |
| | | collected | |
| 00:07 | 00:50 | Patient responds to | Questions and responses stored on |
| | | | PPM |
| | | questions | Indicators of ACS |
| (2 | (2 | Data transmitted to | Data validity checks |
| min) | min) | System Server and | Data retransmissions, when needed |
| | | processed | |
| 00:08 | 00:50 | Automated ECG | Dispatch EMS, if required |
| | | Interpretation | |
| 00:10 | 01:00 | Data available to | Follow-up interpretation of ECG |
| | | Cardiac Tele- | |
| | | consultant | |
| 00:11 | 01:11 | Voice contact with | Follow-up directives to patient |
| | | patient | |
| 00:20 | 01:30 | Data to ED | Option for additional functionality |
| | | | ED Workstation |
| 12:00 | 24:00 | Data to Personal | Update of patient record |
| | | Physician | Updates to patient's personal module |

PIERS Capabilities Summary

The proposed Patient Initiated Emergency Response System, through components and a set of procedures, provides capabilities that:

Equip identified high-risk patients with a small, low-cost device for obtaining 12 lead ECG readings.

Transmit ECG waveforms via any telephone to designated facilities that are equipped to record, interpret, and display the ECG waveform.

Obtain current patient history by interactive responses to programmed questions. Combine with past history retained in the Patient's Personal Module.

Store and forward past and current medical history to designated facilities.

Incorporate an ECG interpretation process that performs automatic ECG interpretation and other support for Cardiac Teleconsultant, who, in cases other than clear-cut myocardial infarction or myocardial ischemia, decides what pathway the patient should take based on clinical and ECG formation.

Transmits all relevant information to the EMS, assuring that the latest patient status and physician recommendations are available to the EMS team.

Has the patient's personal physician receiving all operability and medical data and being responsible for maintaining the information content in the Patient's Personal Module.

Benefits of the example Patient Initiated Emergency Response System discussed herein include:

Diagnostic accuracy is improved over prior art home monitoring devices.

The monitoring device is portable and can be unobtrusively carried by a patient outside the home.

Builds confidence in and familiarity with the PIERS by providing for automated and human guidance via periodic use in non-critical situations.

The critical “time-to-first-call” is reduced by building patient confidence. The EMS will not be dispatched unless the patient is clearly having an infarction or ischemia, or a cardiology specialist dispatches the EMS.

When the EMS is dispatched, patient history and medication information relayed to the EMS team will reduce time for diagnosis and improve emergency treatment at the patient's location and at the ED.

For patients with certain conditions and who are equipped with the Patient's Personal Module, periodic reporting of ECG and recent history will be useful for disease management (and potentially reduce the likelihood of a life threatening cardiac event).

The top-level function of the ECG element of the present invention allows a symptomatic patient, with minimal training and practice, to rapidly obtain an ECG within the confines of the patient's home or office workplace and transmit the ECG to a remotely located physician. A goal for timeliness in Mode 3 of this function is receipt of the ECG by the physician within 15 minutes of the onset of symptoms. There is no specific timeliness goal in Modes 1 and 2. The ECG must contain sufficient data to allow the remote physician to diagnose ACS with a high degree of certainty.

ECG quality adequate to diagnosis ACS indicates a 12-lead system. For some Mode 2 uses, a single bipolar lead configuration may be employed. The rapidity and ease with which a patient can attach electrodes is critical to patient acceptance and the timeline reduction necessary to realize clinical benefit from the system. A goal is to allow electrode placement within 5 minutes from onset of symptoms by a patient who is highly stressed. An electrode placement error of 1 inch is acceptable for limb leads. The system includes means for preventing and/or detecting and correcting limb lead reversal using existing ECG analysis algorithms such as the Marquette Analyzer. Electrode placement errors of less than 0.5 inches are required for precordial (chest) electrodes. In a preferred embodiment the electrodes are reusable to allow for training and practice benefit from Modes 1 and 2. Inexpensive disposable electrodes could also provide this benefit.

Lead Interface

In a preferred embodiment, the lead interface consists of wires which route the ECG signal from the electrodes to signal conditioning within the ECG device. Preferably, the wires should be harnessed and connected in such a way as to add less than 5 seconds of time to the ECG data collection process for a trained and practiced patient under moderate duress of chest discomfort and provide less than 1% attenuation of the ECG signal.

Signal Conditioning

In a preferred embodiment, signal conditioning for the signals from the ECG electrodes is provided. The signal conditioning should have adequate input sensitivity for typical ECG voltages (typically less than ±200 mV) at electrodes, analog to digital sampling at a rate of at least 200 samples per second at an amplitude resolution of 256 steps (8 bits).

Waveform Storage

In a preferred embodiment, the ECG device provides for storage of 10 ECG recordings, 30 seconds in length each in non-volatile memory that can be cleared by a software command (typically issued by a remote data receiver).

Data Processing

In a preferred embodiment, ECG data is processed so as to allow display of a diagnostic quality ECG waveform after transmission to a remote site. Data processing provides for addition of time tags to recordings, checks for data integrity and checks for data validity.

User Interface

In a preferred embodiment, the ECG element supports user controls that originate in the Data Management element. The user interface will allow a patient to initiate a recording after lead placement is complete, initiate data transmission upon connection to the communications service, and provide indications that another ECG recording should be made.

Recorded Questions

In a preferred embodiment, a series of questions used in Modes 1 and 3 operation are stored in memory. After the ECG data is collected these questions are displayed and the patient's responses, which are entered using buttons on the module, are recorded.

ECG Electrodes

The ECG electrodes can comprise a reusable, quick connection to the chest via elastic band, harness, or clothing article that provides a full set of precordial leads. Clear physical indications for limb lead location and precordial array orientation can also be provided, and preferably, no use of conducting gel (to permit reusability and to avoid potential for inter-electrode conduction via gel smearing) is required.

Additionally, in a preferred embodiment, the lead interface should provide for 10-wire cable gathering to a single bundle that connects to the recording device via a single plug.

System Server and Client Modules

The client-server architecture provides the link between the patient and the portions of the medical community being brought to the patient's aid. The System Server receives ECG and patient data as well as the responses to automated questions from the Patient's Personal Module. These data are processed and forwarded to various client modules based on the operating mode indicated in the data. Mode 3 data are also temporarily stored for retrieval by EMS, and/or ED personnel until the patient has been formally received by ED or has been definitively diagnosed as not having ACS.

Performance Specifications—System Server

The primary performance requirements placed on the system server include:

the number of calls it can process simultaneously,

the maximum time to process any call,

the maximum dead time in any one call,

the time to detect server status change, and

the time to redirect call to redundant/backup system.

An order-of-magnitude analysis of the number of Mode 3 calls that the system server will receive on any day based on data from Maryland Emergency Medical Services is in the range of 30 calls per day, and is increased by two orders of magnitude as a worst case to account for Mode 1 and 2 calls as well as a single PIERS server serving the entire State of Maryland. Three thousand calls per day corresponds to approximately two calls per minute. The average duration of each call is mode dependent. Mode 1 calls are expected to last as long as it takes to verify and process the patient information and return a response to the patient (5 minutes estimated). Mode 2 calls are expected to take, on average, approximately as long as Mode 1 given that in some cases the Service Technician will interact with the patient. Mode 3 calls should take approximately twice as long as Mode 2 calls since the Cardiac Teleconsultant may keep the patient on the telephone while EMS providers are responding. Consequently, a conservatively high estimate of an average of 6 calls active at any time is used as a performance requirement.

The time to process any one call is not critical for Modes 1 and 2 operation; however, the patient will perceive excessive time delays as a non-responsive system. Consequently, for Modes 1 and 2, the preliminary performance requirements placed on the maximum time to process any call is 30 minutes and the maximum time the patient has to wait between interactions with the system during a call is 30 seconds.

Time is critical for Mode 3 operation, consequently the maximum time for the System Server to validate the data, process the ECG and then forward the information package to the Cardiac Teleconsultant is 5 minutes.

Reliability of the System Server and communication links is critical to maximizing the benefits of PIERS operation. Consequently, the System Server can include the ability to detect when the system is not operating and roll over calls to a backup system. System status checks can be done every five seconds and calls can be redirected within one second of a non-operating system being detected.

Functional Specifications—System Server

In a preferred embodiment, the following capabilities are resident on the System Server:

Mode determination,

Network link creation and status monitoring with participating organizations,

IP address configuration, control and assignment,

Interface between telephone and internet mediums,

The ability to configure the PIERS to accommodate many combinations of participating organizations, and

Automated ECG interpretation.

In a preferred embodiment, the System Server has two primary purposes: 1) automatic interpretation of ECG and dispatch of EMS provider if warranted, and 2) acting as a router for the information passed between the participants of PIERS. The System Server receives an ECG, interprets it to decide if there is an AMI in progress and if so, dispatches EMS immediately without human intervention. In addition, the System Server maintains a record of the system configuration, attaches the system configuration information to the data set it receives from the patient, and transmits the augmented data set to the next destination dictated by the system configuration.

In a preferred embodiment, the System Server contains dedicated hardware and software that receives calls from the patient, and parses the operating mode from the data in the call. If the data is Mode 3, then the System Server processes the data using the GE/Marquette algorithm and assigns an interpretation based on the algorithm output and a strategy for deducing an AMI based on the output. If an AMI is indicated then the System Server automatically notifies the appropriate EMS provider. In all cases, for Mode 3 operation, the System Server forwards the patient data to the Cardiac Teleconsultant. Once the data has been forwarded, the System Server maintains the connection with the patient to enable voice over communications between the Cardiac Teleconsultant and the patient.

If the operating Mode is 1 or 2, the System Server forwards the patient data to the site providing these services and maintains the connection with the patient to enable voice over communications between the Service Technician and the patient, if the patient is having difficulty operating the PPM. Alternatively, all Mode 1 and Mode 2 operations can be completely automated. Review/call-back capability allows medical personnel to review ECGs and provide feedback to the patient.

A preferred system option is for the patient to communicate via direct phone line to the System Server which, in the preferred configuration, is collocated with the Cardiac Teleconsultant and for all clients to communicate via the internet (or by normal voice phone as a backup).

Further, in one embodiment, the software at the System Server contains a configuration file that identifies the sequence of IP addresses that are used in operating Modes 1 and 2. The configuration file is maintained by a PIERS administrator, who is responsible for the information regarding the participating organizations. The information from an example configuration file for Mode 3 operation is shown in Table 4.

TABLE 4
Conceptual Configuration File: Mode 3

| Destination | IP Address |
|---|---|
| 1. EMS | 723.725.725.725 |
| 2. ER | 723.726.726.726 |
| 3. Service Technician | 723.724.724.724 |

In a preferred embodiment, clients initiate contact directly with the server. This allows for maximum flexibility in configuration and location of the healthcare providers, since only one IP, that of the server, needs to be provided to all clients.

In addition to routing data, the System Server has the responsibility of verifying that the links to the participating organizations are active. The System Server will periodically query each IP address in the configuration file to verify the address is accessible. If the address is not accessible, the System Server will implement a backup configuration.

Performance Specifications—Client Modules

The PIERS has the ability to bring the important parts of the emergency department to the patient in a cost-effective and efficient manner. This is accomplished by integrating cardiac physicians with the EMS providers and Emergency Department services. The client modules discussed below enable the integration.

The client modules are associated with medical care providers linked to PIERS as discussed above. The performance requirements for these modules focus on processing and response times as well as data integrity. Time is most critical for the Cardiac Teleconsultant, consequently the software components comprising that client module will be designed and implemented to minimize processing time and provide the physician an awareness of elapsed time since the Cardiac Teleconsultant module received a data set. In particular, the time to respond to connectivity checks will be less than 1 second.

Data integrity will be insured by keeping a copy of the original data set transmitted by the PPM and providing it to the personal physician who has the sole responsibility to maintain the data on the PPM.

Functional Specifications—Client Modules

The functionality of the client modules is contained in software components that receive, process, display and transmit patient information as each client organization contributes to a response in one of the three modes. The functionality of the client modules will include the following.

Patient Personal Module Diagnostics

This software component analyzes the data set from the PPM to identify abnormalities indicative of malfunctions on the PPM or distortion of the data packets during transmission. This component also identifies abnormalities associated with individual electrode data.

Network Status Display

For options that use the telephone system, this software component monitors the status of the connections between the different client modules. The results are displayed in a window on the client module.

Patient Historical Data Display

This software component displays the medical data that the personal physician has placed on the PPM. Medical data, in a preferred embodiment, comes from four distinct data groups: demographics, medications, medical history including cardiac risk factors, and baseline ECG. The data is organized and displayed in a window.

Patient New Data Display

This software component displays the new ECG transferred from the PPM. The display is similar to the historical data display but include controls to customize the presentation.

ECG Interpretation

This software component uses interpretation algorithms (e.g., the GE/Marquette interpretation algorithm) to process the new ECG data. An ECG results display can also be included.

EMS Provider Screen Display

This software component strips the relevant information from the patient historical data and the Cardiac Teleconsultant's Log and then displays it on the EMS provider display.

Interpretation Log Display

This software component allows the Cardiac Teleconsultant to enter interpretation notes and any other annotation of the patient's ECG. The information is forwarded to others (e.g., EMS, ED and personal physician) as well as entered into a database for tracing and bookkeeping purposes. In addition, this module allows for creating an ECG hardcopy with annotation.

Voice Link to Patient

This software component displays a button that allows the Cardiac Teleconsultant to open a direct voice line with the patient.

Directives Display

This component displays a window that allows the physician to issue a directive for the patient. The following software buttons could be displayed: alert EMS, patient visit ED, patient visit personal physician, re-position electrodes, record another ECG.

PPMData Manager

This software component is used by the personal physician to maintain the information stored in the PPM. The component consists of an editor that allows the personal physician to display and edit the three groups of data stored on the PPM (i.e., medications, medical history, baseline ECG). It also allows the personal physician to issue a request for a data set to the service technician, which is entered on the technician's work list.

Service Technician

This software component consists of a work list of patients that the service technician has to contact and a database of information about personal physicians that the service technician needs in order to provide ECG data.

Automated Questions

In Mode 2 operation this software component generates questions based on patient history and responses to previous questions. The responses are recorded and forwarded to the personal physician as part of the data set.

These software components can be combined to produce the desired functionality for the three operating modes as summarized in Table 5.

TABLE 5
Software Components Assigned to Client Modules

| | Service | Cardiac Tele- | ED/Chest | | Personal |
|---|---|---|---|---|---|
| Client Functionality | Provider | consultant | Pain Ctr. | EMS | Physician |
| OPERATING MODE | 1 and 2 | 3 | 3 | 3 | 1, 2 and 3 |
| 1. Network Status Display | X | X | X | | |
| 2. Patient Historical Data Display | X | X | X | | X |
| 3. Patient New Data Display | X | X | X | | X |
| 4. ECG Interpretation Display | | X | X | | X |
| 5. EMS Provider Screen Display | | X | X | X | |
| 6. Interpretation log Display | | X | X | | |
| 7. Service Technician | X | | | | |
| 8. PPM Diagnostics | X | X | | | |
| 9. Automated Questions | X | | | | |
| 10. Voice Link to Patient | | X | X | | |
| 11. Directives Display | | X | X | | |
| 12. PPM Data Manager | | | | | X |

Decision Support Performance Specifications

The system implements decision support using the information transmitted by the user and the information generated during an automated question and answer session conducted by the decision support system and the user. The capacity requirement for the interactive query process is determined by the maximum number of questions in any one session and the maximum number of questions available for all question set trees. In a preferred embodiment, PIERS will average approximately 10 questions to the patient per Mode 1 session, but in disease-specific Mode 2 question sets, the average will be approximately 20. The total number of questions in the question set tree for each disease will average approximately 50; and, the number of disease-specific question set trees will be approximately 10.

Communication Services Performance Specifications

The system of the present invention provides for universal access by supporting commonly available communications. Current choices include the public switched telephone network (PSTN) by direct dial-up from patient to System Server. Communication between the Cardiac teleconsultant and the other client modules can be performed in a web-enabled fashion using an Internet Service Provider (ISP) as an intermediary. Communications services should preferably add no more than an average of 10 seconds to any one data transfer or patient query session.

Communication Services Functional Specifications

The functions of communications services include the following:

Automatic dialing, call negotiation, user authentication, and data connection between the Patient Personal Module (PPM) to the system server,

Retrieval and transmission of data from the PPM to the System Server,

Ability for a technician and/or physician to converse with a patient without interrupting data communications,

Ability for the System Server to interrogate the patient with scripted questions and for the patient to be able to respond by simple button pushes at the PPM or telephone, and

Automatic disconnection after session completion

Numerous industry examples exist of point-to-point communications used for transmission of cardiac rhythm data over the PSTN. Generally existing point-to-point communications do not allow voice and data to be maintained simultaneously. There are industry examples of self-contained videophones that provide for simultaneous data and voice over a single telephone line and standards exist for doing this. The communications service using a point-to-point approach consists of communications functions distributed between the PPM, the System Server, and the PSTN. The PPM originates calls, provides authentication data, retrieves appropriate data from memory and formats and transmits it. The PPM receives query codes (or analog queries) and transmits patient responses. The System Server auto-answers calls from the PPM, queries for and processes receipt of PPM authentication data, and provides queries or codes for pre-stored queries and receives PPM responses. Both the PPM and System Server respond to degraded line conditions and negotiate to provide optimized data transfer rates. The PSTN provides standardized connectivity between the PPM and the System Server and supports the use of a telephone rotary answer capability at the System Server, so that multiple callers can be serviced simultaneously. This approach requires data be provided to remote users (e.g. Cardiac Teleconsultant, ED, EMS) in a separate step.

A web-enabled approach not only facilitates voice and data (using voice over IP, also known as “internet telephone” approach), but also allows the most flexibility by allowing all facilities—PPM, System Server, EMS, ED and Cardiac Teleconsultant to be simultaneously connected. The web-enabled approach also facilitates future upgrades to connectionless broadband and municipal wireless services as these become increasingly available to the public. The communications service using a web-enabled approach consists of communications functions distributed between the PPM, the System Server, the ISP, and the PSTN. This approach requires not only the PPM functions of point-to-point, but also that the PPM auto-connect via a point-to-point protocol and PSTN to an ISP with a high availability point-of presence. To ensure that attempts to connect are not frustrated by busy signals, multiple access numbers (and/or alternate ISPs) can be programmed into the PPM. Future upgrades would allow the PPM to connect to the ISP using the best technology available to the patient. The ISP provides the interface to other web participants. The ISP can host a dedicated broadband connection between the system server and the Internet. Optimized data transfer negotiation is handled between the PPM and the ISP, and between the ISP and the system server.

To insure a high standard of reliability for the critical patient-system server-Cardiac teleconsultant connection, web-based communications are only used from the Cardiac teleconsultant to the other client modules in the preferred embodiment.

The Emergency Medical Services (EMS) in the United States are organized by states or local jurisdictions (e.g., county or municipality), and consequently, are subject to a wide range of regulations and controls. The net effect is that the protocol used by EMS providers can vary dramatically from location to location. Thus any system designed to bring the ED to the patient and decrease the barriers to using EMS in the case of an ACS must be configurable to accommodate local EMS protocols.

System configuration is facilitated by modular design of the software. The minimum configuration consists of the PPM unit linked to the System Server and the Cardiac Teleconsultant Module. The System Server receives the data from the PPM, does processing focused on ECG interpretation and data validation and forwards the data to the Cardiac Teleconsultant for review and patient disposition. The System Server may dispatch an EMS provider, if not, the teleconsultant may activate a call to EMS, which dispatches the EMS unit.

In order to provide training, advising and administration services to the patient, the minimum functionality can be extended to include a commercial organization (i.e. the service technician) as a client module. This client may be the preferred location of the System Server. The core functionality can also be extended to include an EMS and/or ED client module. Clinical data concerning the patient (historical and ECG) can be transmitted to the EMS provider to assist in their on-site evaluation. Finally, the core functionality can also be extended to include a link to patient's personal physician.

In addition to selection of system modules and communication paths, the functionality of modules would be adapted to local procedures and operational protocols as well. For example, one region may choose to dispatch the EMS based on automatically tested criteria, while others may require that a physician be consulted before alerting the EMS. A brief discussion of the inherent flexibility in the client modules follows.

The EMS mobile unit is in communication with the EMS dispatcher using existing services. The EMS providers can receive patient medical history data (i.e., the EMS screen) via several methods. These include a summary provided by voice over a radio link to data transfer to the mobile EMS unit using radios.

Physicians are directly involved in Mode 3 operation for interpretation of medical data as it is collected. Note that for Mode 3 operation, this interface could be located at the Chest Pain Center, the ED, or a physician's office. It could also be located at the commercial service site where staff medical personnel review the data as it is collected. The Chest Pain Center and hospital ED interfaces are identical.

The process of collecting and analyzing the data (stored ECG and stored medical history) for system operability verification will be done automatically. Since the process is automated, the computer can be located anywhere as long as the communications links exist. Thus configurations that include a private company in one state providing service technician support to Emergency Medical Services personnel in another state are technically possible.

While there has been described herein the principles of the invention, it is to be understood by those skilled in the art that this description is made only by way of example and not as a limitation to the scope of the invention. Accordingly, it is intended by the appended claims, to cover all modifications of the invention which fall within the true spirit and scope of the invention.

1. A patient monitoring system enabling monitoring of patient parameters of a patient from one or more locations remote to said patient, comprising:
patient monitoring equipment;
a server coupled for communication with said patient monitoring equipment;
one or more devices coupled for communication with said server and located in one or more of said locations remote to said patient;
whereby said patient monitoring equipment is configured to operate in a plurality of modes to enable different modes of communication between said patient monitoring equipment and said one or more devices.

2. The patient monitoring system of claim 1, wherein said patient monitoring equipment comprises:
a processing device configured to obtain, store, and transmit to said server, medical data pertaining to said patient.

3. The patient monitoring system of claim 2, wherein said processing device comprises:
a harness containing electrocardiogram (ECG) electrodes and electronics that provide ECG lead measurements;
a personal digital assistant (PDA) coupled to said harness and configured to collect ECG data from said patient;
memory operatively coupled to said PDA for storing collected ECG data;
a communication element operatively coupled to said PDA enabling transmission of collected ECG data to said server.

4. The patient monitoring system of claim 2, wherein said processing device comprises:
a harness containing electrocardiogram (ECG) electrodes and electronics that provide ECG lead measurements; and
and integrated PDA/cellular telephone coupled to said harness and configured to collect ECG data from said patient, store collected ECG data, and transmit collected ECG data to said server.

5. The patient monitoring system of claim 2, wherein said one or more devices coupled for communication with said server comprise at least one of personal computers, PDAs with network connectivity, pagers, cellular telephones, and integrated PDA/cellular telephones.

6. The patient monitoring system of claim 5, wherein said one or more devices coupled for communication with said server are used by health care professionals to access data pertaining to patient parameters monitored by said patient monitoring equipment.

7. The patient monitoring system of claim 1, wherein said plurality of modes of operation include:
a first mode whereby patient parameters are collected using said patient monitoring equipment and are not forwarded to said server.

8. The patient monitoring system of claim 7, wherein said plurality of modes of operation further include:
a second mode whereby patient parameters are collected using said patient monitoring equipment and are automatically forwarded to said server.

9. The patient monitoring system of claim 8, wherein said second mode further includes the automatic storing of said collected patient parameters on said patient monitoring equipment.

10. The patient monitoring system of claim 9, wherein said plurality of modes of operation further include:
a third mode whereby patient parameters are collected using said patient monitoring equipment and are automatically forwarded to said server, and whereby one or medical professionals are automatically alerted of said automatic forwarding of said patient parameters when analysis of said patient parameters indicate a predetermined condition.

11. The patient monitoring system of claim 9, wherein said plurality of modes of operation further include:
a third mode whereby patient parameters are collected using said patient monitoring equipment and are automatically forwarded to said server, and whereby one or medical professionals are automatically alerted of said automatic forwarding of said patient parameters based upon an action taken by the user of said patient monitoring equipment.

12. The patient monitoring system of claim 11, wherein said action taken by the user comprises sending an alert to said server indicating that the patient is experiencing symptoms requiring emergency care.

13. The patient monitoring system of claim 3, wherein said harness comprises:
a support element worn by the patient;
a plurality of electrodes situated in said support element and coupleable to said processing device;
wherein said plurality of electrodes are situated properly for monitoring of said patient parameters when said harness is worn by the patient.

14. The patient monitoring system of claim 13, wherein said electrodes are non-adhesive, reusable, gel-free electrodes.

15. The patient monitoring system of claim 14, wherein said harness provides 12-lead ECG capability.

16. A method for enabling monitoring of patient parameters of a patient from one or more locations remote to said patient, comprising the steps of:
collecting patient parameters from said patient with patient monitoring equipment;
providing a server coupled for communication with said patient monitoring equipment;
providing one or more devices coupled for communication with said server and located in one or more of said locations remote to said patient;
configuring said patient monitoring equipment to operate in a plurality of modes to enable different modes of communication between said patient monitoring equipment and said one or more devices; and
communicating said collected patient parameters from said patient monitoring equipment to said server in at least one of said plurality of modes.

17. The method of claim 16, wherein said step of collecting patient parameters comprises the steps of:
obtaining, storing, and transmitting to said server, medical data pertaining to said patient.

18. The method of claim 17, wherein said step of collecting patient parameters further comprises the steps of:
connecting to said patient a harness containing electrocardiogram (ECG) electrodes and electronics that provide ECG lead measurements;
operating a personal digital assistant (PDA) coupled to said harness and configured to collect ECG data from said patient;
storing said collected ECG data in a memory operatively coupled to said PDA; and
transmitting said stored ECG data to said server via a communication element operatively coupled to said PDA enabling transmission of collected ECG data to said server.

19. The method of claim 16, wherein said plurality of modes of operation include:
a first mode whereby patient parameters are collected using said patient monitoring equipment and are not forwarded to said server.

20. The method of claim 19, wherein said plurality of modes of operation further include:
a second mode whereby patient parameters are collected using said patient monitoring equipment and are automatically forwarded to said server.

21. The method of claim 20, wherein said second mode further includes the automatic storing of said collected patient parameters on said patient monitoring equipment.

22. The method of claim 21, wherein said plurality of modes of operation further include:
a third mode whereby patient parameters are collected using said patient monitoring equipment and are automatically forwarded to said server, and whereby one or medical professionals are automatically alerted of said automatic forwarding of said patient parameters when analysis of said patient parameters indicate a predetermined condition.
